(19) United States
(12) Patent Application Publication
(10) Pub. No.: US 2025/0121158 A1
(43) Pub. Date: Apr. 17, 2025

(54) ATRAUMATIC CATHETER

(71) Applicant: THE GENERAL HOSPITAL CORPORATION, Boston, MA (US)

(72) Inventors: Graham Lieberman, Boston, MA (US); Hersh Bendre, Boston, MA (US); Jonathan Kusner, Boston, MA (US); Francis McGovern, Boston, MA (US); Amy Hao, Boston, MA (US); Avik Som, Boston, MA (US); April Anlage, Boston, MA (US); Hilary Gallin, Boston, MA (US); John Di Capua, Boston, MA (US)

(21) Appl. No.: 18/704,670

(22) PCT Filed: Oct. 24, 2022
(86) PCT No.: PCT/US2022/047571

§ 371 Date: Apr. 25, 2024

Related U.S. Application Data
(60) Provisional application No. 63/271,296, filed on Oct. 25, 2021.

(51) Int. Cl. A61M 25/00; A61M 25/10

(52) U.S. Cl. CPC A61M 25/0017; A61M 25/1011; A61M 2025/1068; A61M 2025/1072; A61M 2025/1084; A61M 2205/0216; A61M 2210/1089

(57) ABSTRACT

An atraumatic catheter is provided. The catheter has an inflation section and a drainage section. At least one balloon is disposed in the inflation section and is offset from the central longitudinal axis of the catheter shaft. The length of the balloon in an actuated state is greater than the length of the balloon in a non-actuated state while the width of the balloon remains substantially the same in an actuated state and a non-actuated state.

### CROSS-REFERENCE TO RELATED APPLICATIONS

This application claims the benefit of U.S. Provisional Application Ser. No. 63/271,296, filed 25 Oct. 2021, entitled “SAFECATH, AN ATRAUMATIC CATHETER INTERNALIZATION MECHANISM”. The entirety of this application is incorporated by reference for all purposes.

### TECHNICAL FIELD

The present disclosure relates to an atraumatic urinary catheter.

### BACKGROUND

Urinary catheterization is a common medical procedure with a multitude of uses. While catheterization is generally fairly simple in female patients given a relatively short and straight urethra, the acute angle bend of the prostatic urethra in men makes catheterization in males far more challenging. Flexible catheters have existed for some time to accommodate the prostatic urethral bend. An initial design was a segmented metal tube connected with wire hinges designed in a manner such that the hinges would not traumatize the urethra on insertion and made male catheterization substantially easier and more comfortable.

With the advent of vulcanized rubber, indwelling flexible urinary catheters were developed and an initial design was a double lumen catheter with one lumen allowing drainage of the bladder into collection tubing and the other lumen blindly ending in a radially expanding balloon located at the tip of the catheter that could be inflated once inside the bladder. Such an indwelling urethral catheter (known as a “Foley catheter”) allowed single time placement with continuous bladder drainage for days to weeks thereafter. The design has remained largely unchanged over the past 90 years with only small modifications to tip shape or inclusion of additional lumens available in specialty catheters. A balloon at the catheter tip inflated through a lumen connected to an external balloon port is the mainstay internalization mechanism of nearly every urinary catheter, as well many other indwelling lines/tubes such as percutaneous gastrostomy tubes and endotracheal tubes.

With nearly 10 million indwelling urinary catheters placed in the U.S. on an annual basis, Foley catheterization is second only to IV placement as the most common procedure patients experience. Up to 25% of patients admitted to the hospital will have a urinary catheter placed during their stay and that number increases to over 60% for critically ill patients. Furthermore, over 10% of nursing home occupants are catheterized. With such a large segment of the population undergoing urinary catheterization each year, the complications of the procedure have impacts on population health and healthcare spending. A feared complication of urinary catheterization is the catheter-associated urinary tract infection, or CAUTI, which is responsible for over 1% of in-hospital deaths.

However, the design of the Foley catheter itself is responsible for hundreds of thousands of injuries each year due to catheter-induced trauma, or CIT. CIT is an under-recognized problem compared to CAUTI, even though CIT requiring intervention has a higher incidence than symptomatic CAUTI. CIT generally occurs in three different but related injury patterns. The first type is false passage creation, which is an obstruction of some kind (typically benign prostatic hyperplasia, aka BPH, or urethral stricture) preventing the catheter tip from curving within the prostatic urethral bend. The force of insertion directs the catheter tip through the urethral wall creating a “false passage” beyond the urethra into surrounding tissue. The second type is intraurethral inflation, where the catheter balloon is inflated while still within the urethra, typically because the tip of the catheter is obstructed from passing into the bladder. This is often associated with false passage formation (a catheter tip is typically false passaged, which itself is injurious, and then the balloon is inflated, dilating and disrupting the urethra and surrounding tissue). The third type is traumatic removal, where the catheter balloon is properly inflated within the bladder, but then the catheter is forcefully removed with the balloon inflated causing injury and possible disruption to the bladder neck or urethra, typically due to catheter bags being anchored to hospital beds when patients walk away or unstable patients traumatically self-removing the catheter.

There are procedural and design changes to Foley catheter insertion that have somewhat decreased the frequency of these injuries, such as use of lubricant, use of a coude tip (curve catheter tip that angles the catheter along the trajectory of the prostatic bend), and procedural requirements that balloons not be inflated until a catheter is “hubbed” (inserted all the way to ensure the balloon segment is not in the urethra) with return of urine. These practices are beneficial but there is substantial variability in their use.

These injuries can impact patient health and healthcare spending because up to 78% of CIT results in a long-term urethral stricture disease. These strictures, if untreated, can ultimately lead to voiding dysfunction, bladder injury, chronic renal disease, and increased risk of UTIs. The treatments for urethral strictures range from regular intermittent catheterization to surgical dilation, reconstruction, or urethral diversions. There are financial and non-financial costs associated with these events (such as decreased patient quality of life).

The nature of many of these injuries, and the more injurious of them, is inherent to the existence of the radially expanding internalization balloon that defines the Foley catheter. A standard Foley catheter has a 10 mL roughly spherical balloon around a 16 French drainage tube (“French” is the standard measurement of catheter size, wherein the French number is 3× the diameter in millimeters). The balloon when properly inflated is approximately 90-95 French. On average, the intrapelvic urethra in men is between 26-35 French. As such, if a catheter balloon is inflated inside a urethra, the urethra has typically been dilated to the point of traumatic distention or frank disruption, leading to CIT and associated sequelae. In addition, when a catheter with an inflated balloon is forcefully removed, the bladder neck and urethra are similarly dilated to the point of disruption.

There exist catheter designs that have been manufactured or patented that attempt to solve the problem of CIT. The prime urologic example is a catheter with a single lumen catheter that utilizes flared wings at the tip that resist deformation (but can be deformed with enough force). It is inserted with a stylet placed through the middle of the lumen to straighten out the wings. It was historically used for suprapubic catheterization (SPT—percutaneous drainage of the bladder through the low abdominal wall) or nephrostomy tubes (PCN—percutaneous drainage of the kidney through the flank). They are poorly tolerated as urethral catheters because the material constraints of latex/silicone make it difficult to build a catheter tip that can resist deformation to remain indwelling but have wings perfectly coapt for smooth insertion through the sensitive urethral tissue. Furthermore, removal is similarly uncomfortable or difficult as it either requires extra force or insertion of a stylet to straighten the wings. Their use as SPT or PCN has thus fallen out of favor in exchange for either percutaneous Foley catheters or pigtail catheters. These catheters are now typically reserved for use as temporary rectal tubes or ostomy catheters.

Pigtail catheters are a mainstay of interventional radiology drainage procedures. The catheter is a single lumen catheter with a string tie that wraps longitudinally around the wall of the catheter and runs through the lumen and out of it at the proximal end. Pulling the string tie will thus bowstring the tie and subsequently bend the catheter tip into an arc around, roughly in the shape of a cartoon pig's tail. The string tie is then held in place externally with a simple plastic clamp. The catheters are typically inserted using fluoroscopy and wire guidance, which limits their utility in a bedside nursing procedure. Furthermore, when the string tie is clamped in place, the pigtail does not uncurl. Traumatic removal of the pigtail without first cutting the string can cause significant tissue damage.

Similar to pigtail catheters are J-stents, commonly used in urology for ureteral stenting. A J-stent is a single lumen catheter with a J-shaped curl at one of both ends that can be straightened out with a modicum of force. These are typically placed in the ureter to drain a ureteral obstruction and are similarly typically placed using fluoroscopy and wire guidance. J-stents have the advantage of being able to resist removal up to a point, and then the J-curve deforms and straightens when pulled out, which could be beneficial regarding traumatic removal, but is logistically complicated as it would require a stylet or wire for insertion (and would be desirable, though not required, for removal in order to minimize).

A safety syringe has been described in the literature, which is a syringe with a pop-off valve that causes redirection of fluid if the pressure inside the balloon lumen increases above a threshold. This is a mechanism that could certainly help prevent intraurethral balloon inflation if the pop-off pressure were calibrated appropriately. This is extrinsic to the catheter, so would not prevent traumatic removal injuries. Additionally, the emphasis on pressure ignores that the injurious factor is the ratio of balloon diameter to maximal urethral diameter, which could occur at different pressures in different catheter sizes and in different patients.

There are several patents for urethral catheter designs (or accessories) specifically aimed at decreasing CIT. For example, EP U.S. Pat. No. 1,305,075B1 describes a catheter with a flanged or bulbous distal tip that, upon application of an axial force on the proximal end, causes a conformational change of the distal tip to straighten it. While this catheter design could decrease catheter trauma, it requires a metal stiffener ribbon and a braided prostatic metal stent to transmit force along the length of it, which increases complexity and cost. It additionally radically changes the catheter insertion methodology, which is an ingrained standard across the entire medical community and has been for decades. Consequently, such a move away from current insertion protocols could substantially hinder adoption.

U.S. Pat. No. 8,939,962 describes a catheter with a separable drainage lumen wall that, with a longitudinal application of force, the drainage lumen will pull apart inside the balloon causing the balloon to rapidly drain into the catheter lumen. This would be useful at preventing traumatic removal injury. It is unclear how useful this would be at preventing intraurethral balloon inflation injuries, as the pressure would certainly increase in the balloon relative to intravesical inflation, but there would not be the same longitudinal force to separate the drainage lumen in this regard. This furthermore can cause a failure of the device, which is more desirable than patient injury, but also means any troubleshooting of improper catheter placement/withdrawal requires new catheter insertion.

### SUMMARY

An atraumatic catheter is provided herein. In an aspect, a catheter has a non-actuated state and an actuated state. The catheter can comprise a catheter shaft having a central longitudinal axis, a distal actuating portion, a proximal portion, opposing first and second outer walls, and an inner wall between the opposing first and second outer walls. A drainage section can comprise a drainage lumen between the inner wall and the first outer wall and a drainage eyelet can be disposed in the drainage section. The catheter can include an inflation section comprising an inflation lumen between the inner wall and the second outer wall. A balloon can be disposed at the distal actuating portion of the inflation section and can be in fluid communication with the inflation lumen. The balloon can be offset from the central longitudinal axis of the catheter shaft and can have a length and a width. The length of the balloon in an actuated state can be greater than the length of the balloon in a non-actuated state while the width of the balloon can remain substantially the same in an actuated state and a non-actuated state.

Inflation of the balloon can cause a conformational change of the catheter that resists removal of the catheter from the bladder without substantially increasing the diameter of the catheter. In the context of a urinary catheter, the catheter can be configured to not expand beyond the diameter of a typically-sized urethra.

### BRIEF DESCRIPTION OF THE DRAWINGS

FIG. 1 is a cross-sectional view of a catheter according to an aspect of the present disclosure.

FIG. 2A is a schematic view of the internal components of a distal actuating portion of a catheter shaft of a catheter in a non-actuated state according to an aspect of the present disclosure.

FIG. 2B a schematic view of the internal components of the distal actuating portion of FIG. 2A in an actuated state of the catheter.

FIG. 3A is a schematic view of the internal components of a distal actuating portion of a catheter shaft of a catheter in a non-actuated state according to an aspect of the present disclosure.

FIG. 3B is a schematic view of the internal components of the distal actuating portion of FIG. 3A in an actuated state of the catheter.

FIG. 4A is side view of a distal actuating portion of a catheter shaft of a catheter in a non-actuated state according to an aspect of the present disclosure.

FIG. 4B is a side view of the distal actuating portion of FIG. 4A in an actuated state of the catheter.

FIG. 5A is a schematic view of the internal components of a distal actuating portion of a catheter shaft of a catheter in a non-actuated state according to an aspect of the present disclosure.

FIG. 5B is a schematic view of the internal components of the distal actuating portion of FIG. 5A in an actuated state of the catheter.

FIG. 6A is a schematic illustration of the internal components of a distal actuating portion of a catheter shaft of a catheter in a non-actuated state according to an aspect of the present disclosure.

FIG. 6B is a schematic illustration of the internal components of the distal actuating portion of FIG. 6A in an actuated state of the catheter.

FIG. 7A is a schematic view of the distal actuating portion of a catheter shaft of a catheter in a non-actuated state according to an aspect of the present disclosure.

FIG. 7B is a schematic view of the distal actuating portion of FIG. 7A in an actuated state of the catheter.

FIG. 8A is a schematic view of the internal components of a distal actuating portion of a catheter shaft of a catheter in a non-actuated state according to an aspect of the present disclosure.

FIG. 8B is a schematic view of the internal components of the distal actuating portion of FIG. 8A in an actuated state of the catheter.

FIG. 9A is a schematic view of the internal components of a distal actuating portion of a catheter shaft of a catheter in a non-actuated state according to an aspect of the present disclosure.

FIG. 9B is a schematic view of the internal components of the distal actuating portion of FIG. 9A in an actuated state of the catheter.

FIG. 10A is a schematic view of a distal actuating portion of a catheter shaft of a catheter in a non-actuated state according to an aspect of the present disclosure.

FIG. 10B is a schematic view of the distal actuating portion of FIG. 10A in an actuated state of the catheter.

FIG. 11 is a perspective view of the distal actuating portion of the catheter shaft of a catheter according to an aspect of the present disclosure.

FIG. 12 is a perspective view of the distal actuating portion of the catheter shaft of a catheter according to an aspect of the present disclosure.

FIG. 13 is a perspective view of the distal actuating portion of the catheter shaft of a catheter according to an aspect of the present disclosure.

FIG. 14 is a perspective view of the distal actuating portion of the catheter shaft of a catheter according to an aspect of the present disclosure.

### DETAILED DESCRIPTION

As used herein with respect to a described element, the terms “a,” “an,” and “the” include at least one or more of the described element(s) including combinations thereof unless otherwise indicated. Further, the terms “or” and “and” refer to “and/or” and combinations thereof unless otherwise indicated. By “substantially” is meant that the distance, shape, or configuration of the described element need not have the mathematically exact described distance, shape, or configuration of the described element but can have a distance, shape, or configuration that is recognizable by one skilled in the art as generally or approximately having the described distance, shape, or configuration of the described element. As such “substantially” refers to the complete or nearly complete extent of a characteristic, property, state, or structure. The exact allowable degree of deviation from the characteristic, property, state, or structure will be so as to have the same overall result as if the absolute characteristic, property, state, or structure were obtained. The terms “first,” “second,” etc. are used to distinguish one element from another and not used in a quantitative sense unless indicated otherwise. Thus, a “first” element described below could also be termed a “second” element. A component “connected to,” “operably connected to,” “disposed adjacent to,” “disposed between,” “disposed on,” “between,” “located at” another component can have intervening components between the components so long as the catheter can perform the stated purpose. The sequence of operations (or steps) is not limited to the order presented in the claims or figures unless specifically indicated otherwise. As used herein a “patient” includes a mammal such as a human being. Although the drawings show certain elements of a catheter in combination, it should be noted that such elements can be included in other embodiments or aspects illustrated in other drawings. In other words, each of the disclosed aspects and embodiments of the present disclosure may be considered individually or in combination with other aspects and embodiments of the disclosure.

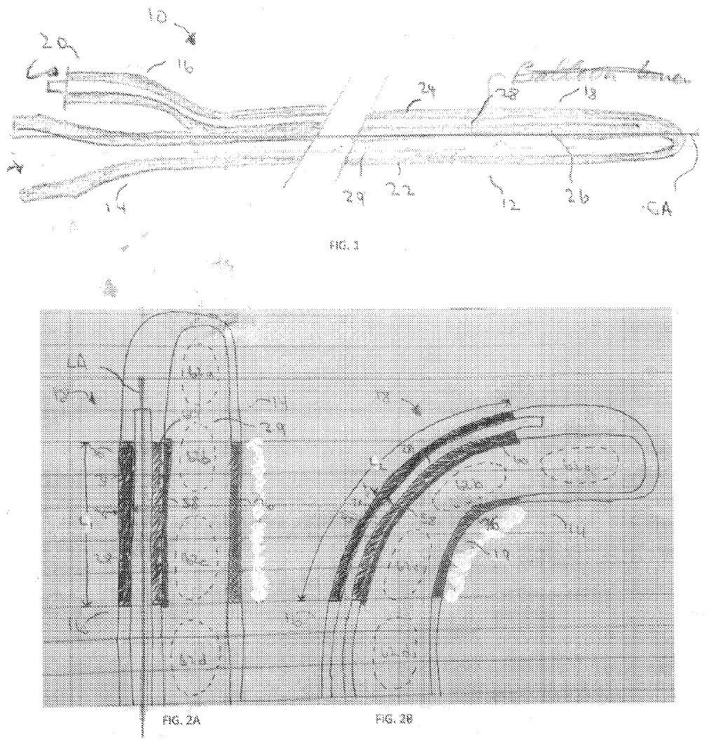

In an aspect and with reference to FIGS. 1 and 2, catheter 10 is provided that can assume a non-actuated state and an actuated state. Catheter 10 can include catheter shaft 12 having a central longitudinal axis CA and can comprise inflation section 16 and drainage section 14. In particular, catheter shaft 12 can comprise distal actuating portion 18, proximal portion 20, opposing first and second outer walls 22 and 24 respectively, and inner wall 26 between opposing first and second outer wall 22 and 24 respectively. In an actuated state, the distal actuating portion can be configured to bend in a manner that creates an inner curvature 19 of the distal actuating portion. In certain aspects, the distal actuating portion comprises the distal third to distal quarter of the catheter shaft.

Drainage section 14 can comprise drainage lumen 29 between inner wall 26 and first outer wall 22. A drainage eyelet(s) 62 can be disposed in drainage lumen 29 to allow drainage of fluid from the catheter. The drainage eyelet(s) can have different orientations, shapes, and distribution patterns so long as the drainage eyelet(s) can allow fluid to drain from the catheter. For example, the drainage eyelet(s) be located distal, proximal and/or within the distal actuating portion. In certain aspects, the draining section includes a plurality of drainage eyelets. A distal-most drainage eyelet can serve as the first drainage point and initial indicator to the user that the catheter is draining and therefore is in the correct location. A proximal-most drainage eyelet can allow sufficient drainage even if the more distal drainage eyelets are partially occluded by actuation of the catheter or if the catheter has not been fully inserted into the bladder (e.g. the tip of the catheter is in the bladder but the shaft is in the urethra). In certain aspect, all of the drainage eyelets are located distal to the distal actuating portion of the catheter shaft and in certain aspects, all of the drainage eyelets are located proximal to the distal actuating portion of the catheter shaft.

Inflation section 16 can comprise inflation lumen 28 between inner wall 26 and second outer wall 24. A balloon 30 in fluid communication with inflation lumen 28 can be located at distal actuating portion 18 and can be defined by outer layer 31 facing away from the drainage section and inner layer 60 facing towards and adjacent to the drainage section. Balloon 30 can be offset from central longitudinal axis CA of catheter shaft 12 and can assume an axially lengthened configuration in an actuated state as illustrated in FIG. 2B and a substantially non-radially expanded configuration in an actuated state and a non-actuated state as illustrated in FIG. 2B and FIG. 2A, respectively. In particular, the balloon can have a length L and a width W. The length L₂ of the balloon in an actuated state (FIG. 2B) can be greater than the length Li of the balloon in a non-actuated state (FIG. 2A). However, the width W of the balloon can remain substantially the same in an actuated state (W₂) and a non-actuated state (W₁). The width/diameter of the balloon in an actuated state can be limited such that the width/diameter of the catheter is less than 26 Fr, for example. The length and width of the portion 76 of the catheter shaft opposite the inflation section at the distal actuating portion can remain substantially the same in a non-actuated state and an actuated state. As such, the inflation section at the distal actuating section can elongate while the opposite side can not. This can cause the distal actuating portion to curl towards the drainage section of the distal actuating portion. The balloon(s) can be configured such that it expands differentially along the longitudinal axis LA of the inflation section while limiting radial expansion. Specifically, in an unactuated state the length of second wall 24, inner wall 26 and first wall 22 can be substantially equal. In an actuated state, the length of second wall 24 can be greater than the length of inner wall 26 which can be greater than the length of first wall 22. In both an actuated state and an un-actuated state, the diameter (or circumference) of the actuating distal portion remains the substantially the same.

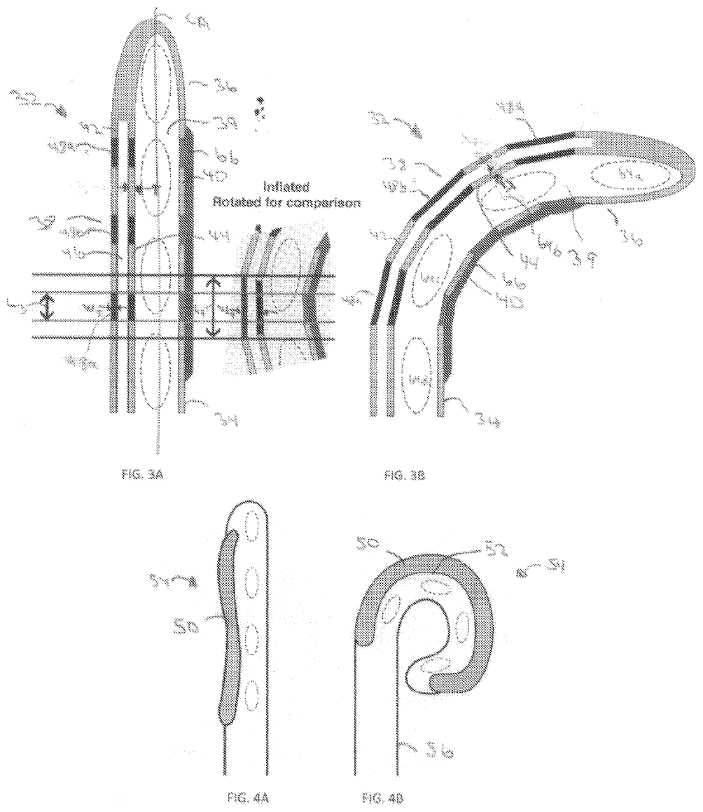

A catheter can comprise a single balloon as illustrated in FIG. 2 or a plurality of balloons. For example and with reference to FIG. 3, a catheter can comprise a catheter shaft 34 having drainage section 36 and inflation section 38. Similar to the embodiment illustrated in FIG. 2, catheter shaft 34 can comprise distal actuating portion 32, a proximal portion (not shown), opposing first and second outer walls 40 and 42 respectively, and inner wall 44 between opposing first and second outer wall 40 and 42 respectively. Drainage section 36 can comprise drainage lumen 39 between inner wall 44 and first outer wall 40. A drainage eyelet 64, including a plurality of drainage eyelets can be disposed within the drainage lumen similar to the embodiment illustrated in FIG. 2. Inflation section 38 can comprise inflation lumen 46 between inner wall 44 and second outer wall 42. A plurality of balloons 48, such as, for example, a series of balloons in fluid communication with inflation lumen 46 and positioned substantially parallel to the central longitudinal axis CA of the catheter shaft can be located at distal actuating portion 38 offset from central longitudinal axis CA of catheter shaft 12 and assuming an axially lengthened configuration in an actuated state as illustrated in FIG. 3B and a substantially non-radially expanded configuration in an actuated state and a non-actuated state as illustrated in FIG. 3B and FIG. 3A, respectively. In particular, each of the plurality of balloons 48 can have a length L and a width W. The length La of each of the balloons in an actuated state (FIG. 3B) can be greater than the length L₃ of the balloon in a non-actuated configuration (FIG. 3A). However, the width W of the balloons can remain substantially the same in an actuated state (W₄) and a non-actuated state (W₃).

A catheter can comprise a balloon(s) disposed within the interior of the inflation section at the distal actuating portion as illustrated in FIGS. 2 and 3 or disposed on the exterior of the inflation section at the distal actuating portion as illustrated in FIG. 4 (inflation lumen and drainage lumen not shown). In particular, FIG. 4 illustrates a balloon 50 disposed on outer curvature 52 of the distal actuating portion 54 of catheter shaft 56 of a catheter. Although FIG. 4 illustrates a single balloon, the catheter could include a plurality of balloons.

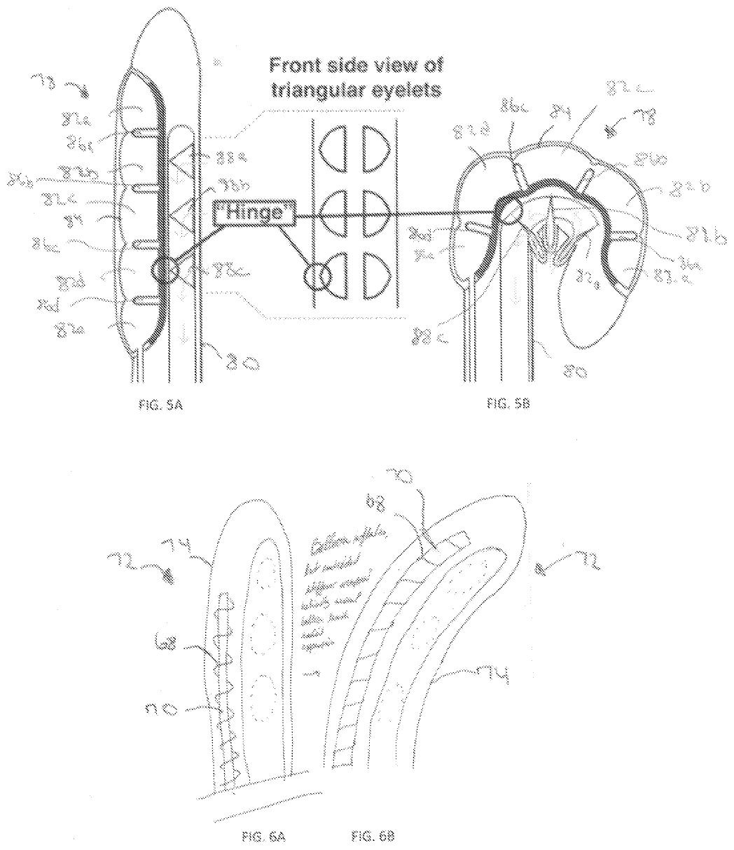

Referring to FIG. 5, in certain aspects, distal actuating portion 78 of catheter shaft 80 of a catheter can include a balloon(s) 84 with semi-segmental sections 82 disposed within the inflation section. FIG. 5A illustrated the catheter in a non-actuated state and FIG. 5B illustrates the catheter in an actuated state. The balloon(s) is semi-segmented such that a septum/septae 86 are disposed within balloon(s) 84 to allow balloon(s) 84 to angle with a given hydraulic force without moving fluid from one balloon section 82 to another (Laplace's law). In addition to allowing drainage of fluid, drainage eyelets 88 can also be used in this (or any other embodiments) as static hinges to further allow bending of the catheter tip.

A benefit of this embodiment is that the catheter can be inserted as a straight (or coude tip, for example) flexible catheter and the distal actuating portion of the catheter shaft can then bend much like a shepherd's crook to resist removal, but there is no portion of the catheter that requires inflation to the same degree of radial expansion as a standard internalization balloon of a Foley catheter. This can prevent both intraurethral inflation and traumatic removal injuries because the force generated by the angulation induced by inflating the balloon(s) can be titrated to a lower threshold than is required to injure tissue. For example, if the balloon(s) were inflated inside the urethra, the diameter at any given point of the catheter may remain less than the diameter of the urethra, so no dilation of a patient's anatomy would occur. The force generated by the inflated balloon(s) would be distributed along the distal actuating portion of the catheter, which would angle the catheter tip against tissue but would create less force against the tissue compared to a typical Foley catheter. If the catheter were inflated in the bladder and pulled out, the catheter could similarly uncurl and slide out. The force and angulation parameters (how “stiff” the shepherd's crook is) of the balloon(s) can be controlled such that the amount of force the catheter can generate is enough to resist removal during typical patient activity (e.g. such as walking) but would “give way” if an otherwise traumatic-Foley-removal force were incurred.

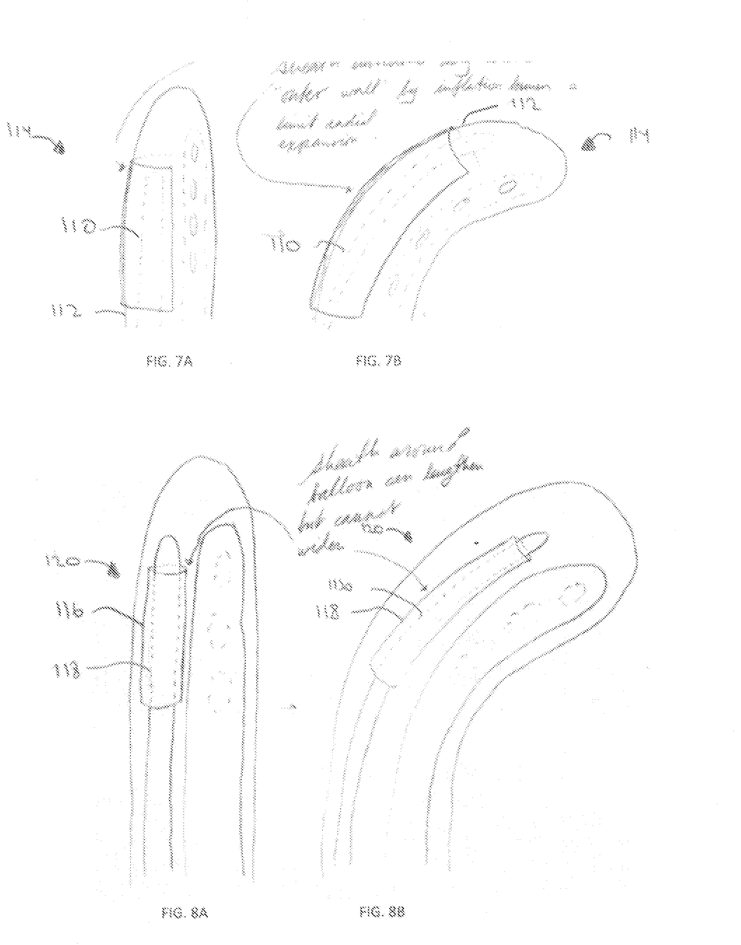

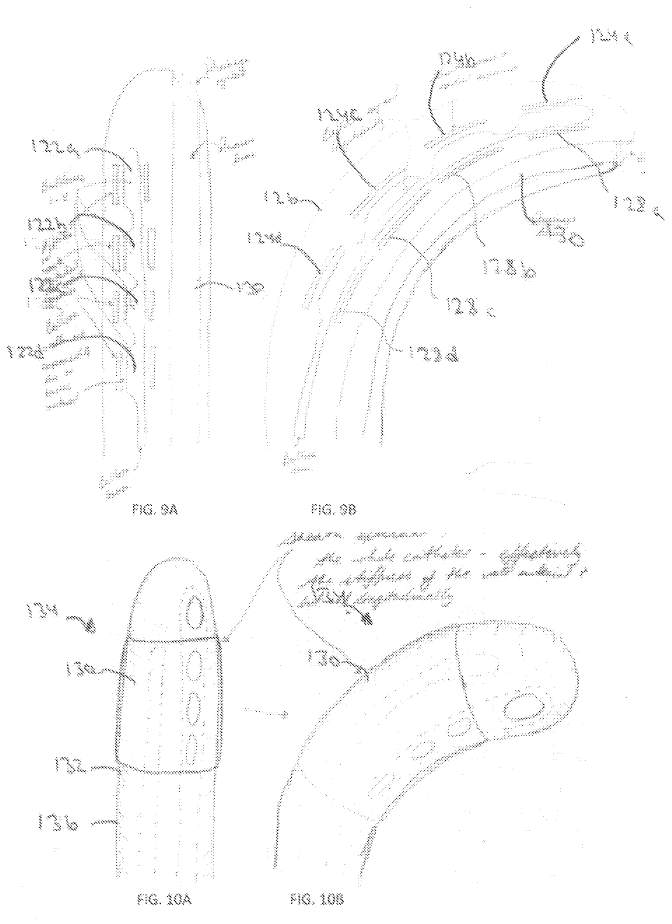

Several properties of the catheter can give rise to the balloon(s) of the catheter assuming an axially lengthened configuration in an actuated state and a substantially non-radially expanded configuration in an actuated and a non-actuated state. For example, the inner wall (or any other wall adjacent to the balloon(s)) can have a thickness T (see FIG. 3) sufficient to promote axially lengthening of the balloon(s) while limiting or preventing radial expansion of the balloon(s). Alternatively or additionally and with reference to FIG. 2A, a semi-rigid stiffener 58 can be disposed adjacent to or embedded within the side 60 of balloon 30 closest to the drainage section at the distal actuating section to achieve the same property. Alternatively or additionally and with reference to FIG. 7, a semi-rigid stiffener 110 can be disposed adjacent to or embedded within second outer wall 112 at distal actuating portion 114 (the wall adjacent to the inflation section). FIG. 7A illustrates the distal actuating portion when the catheter is in an un-actuated state and FIG. 7B illustrates the distal actuating portion when the catheter is in an actuated state. Alternatively or additionally and with reference to FIG. 8, a semi-rigid or elastic sheath 116 can be selectively disposed about or embedded within the inflation section 118 that includes at least the distal actuating portion 120 of the catheter shaft. FIG. 8A illustrates the distal actuating portion when the catheter is in an un-actuated state and FIG. 8B illustrates the distal actuating portion when the catheter is in an actuated state. Alternatively or additionally and with reference to FIG. 9, in the case of a plurality of balloons or balloon segments 122, semi-rigid stiffeners 124 can be disposed on the side of each of balloons or balloon segments 122 closest to the exterior of the catheter 126 and semi-rigid stiffener 128 can be disposed on the side closest to drainage section 130. Such stiffeners can promote longitudinal extension over radial expansion. FIG. 9A illustrates the distal actuating portion when the catheter is in an un-actuated state and FIG. 9B illustrates the distal actuating portion when the catheter is in an actuated state. Alternatively or additionally and with reference to FIG. 10, a semi-rigid sheath 130 can be disposed on the external surface 132 of distal actuating portion 134 of catheter shaft 136 effectively increasing the stiffness of the external surface at that section radially but not longitudinally. FIG. 10A illustrates the distal actuating portion when the catheter is in an un-actuated state and FIG. 10B illustrates the distal actuating portion when the catheter is in an actuated state. Alternatively or additionally and with reference to FIG. 6, a coil, spring 68, ring(s) or similar structure can be disposed about the inflating section at the distal actuating portion 72 or individual balloon(s) 70, the coil, spring 68 ring(s) or similar structure being configured to stretch longitudinally to allow axial lengthening of the balloon(s) but limiting or preventing radial expansion of the balloon(s) as seen by a comparison of FIG. 6A, which illustrates catheter shaft 74 in a non-actuated state, and FIG. 6B, which illustrates catheter shaft 74 in an actuated state. Such structures and material features allow the balloon(s) to increase in length but not width (and may also help prevent the balloon(s) from expanding into, and thus possibly occluding, the drainage lumen). Thus, when the balloon is inflated, it lengthens but does not widen. This maintains the cross-sectional diameter (so the balloon does not dilate the catheter), but lengthens one side of the catheter promoting the curling of the catheter at that segment. Other structures and properties could also be incorporated into the catheter to limit radial expansion of the balloon(s), promote axial lengthening of the balloon (s.), and ensure patency of the drainage lumen.

In an actuated state of catheters as disclosed herein, instillation of fluid into the inflation lumen causes the balloon(s) to expand but given the offset position of the balloon(s) relative to the central longitudinal axis of the catheter shaft and the axial lengthening of the balloon(s), the balloon(s) inflation causes one side of the catheter to lengthen relative to the other. This causes inner bending or curling of the catheter, wherein the catheter bends at the level of the balloon(s). In other words, inflation of the balloon(s) causes one longitudinal half of the catheter to “accordion” open relative to the other half, bending the length of catheter at that level away from the balloon(s). As such, the catheter tip can curve within the bladder or body cavity prostatic urethral bend of a patient or other body cavity. In certain aspects, drainage eyelets can be disposed proximal, distal or at the same level as the inner bending or curling of the catheter to allow drainage on insertion and after deployment. A catheter can include a semi-rigid longitudinal spine 66 (such as, for example, a tensile cord) disposed on the catheter shaft opposite of the inflation section at the distal actuating portion to potentiate this conformation change. In particular, spine 66 can promote the specific direction of curling by limiting or preventing curling in the opposite direction (e.g. limiting or preventing the distal actuating portion from curling towards the inflation section but permitting the distal actuating portion to bend towards the drainage section of the distal actuating portion). In other words, such a spine can facilitate formation of a catheter with an inner curvature at the distal actuating portion as depicted in FIGS. 2B, 3B, and 4B.

In a traditional Foley catheter, the retention mechanism in the bladder is the expanded diameter of the balloon which is caused by increased pressure inside the balloon. The retainability is directly correlated to the diameter of the balloon, which is directly correlated to the balloon pressure. This occurs because Foley catheter balloons are roughly spherical or toroidal and thus rotationally symmetric. The primary way to increase the dimensionality of the Foley catheter is to expand the balloon in a radially symmetric manner.

Catheters as disclosed herein can remove these direct correlations in that the balloon(s) are not radially symmetric but rather offset from the central longitudinal axis of the catheter shaft and lengthen axially in an actuated state. An increase in pressure in the balloon(s) does not cause radial expansion of the balloon, but rather elongation of the balloon in a substantially parallel direction to the central longitudinal axis of the catheter shaft but offset from the central longitudinal axis of catheter shaft. This can cause the catheter tip to bend, thus increasing the dimensionality of the catheter compared to a Foley catheter but without substantially increasing the cross-sectional area at any point along the catheter's length.

The non-radially-expanding retention feature of the balloon(s) can allow high pressures to be achieved in the balloon(s) without translating the resultant hydraulic forces perpendicularly to the walls of the urethra or bladder neck of the patient or the wall of another body cavity. This means inflation in the insertion track could be largely atraumatic. This further can allow the catheter to be straightened despite an inflated balloon(s), allowing for largely atraumatic removal with enough force being applied. The non-radially-expanding retention balloon(s) is thus able to internalize a catheter and keep it in place up to a threshold below the level of trauma, thus creating a non-traumatic catheter.

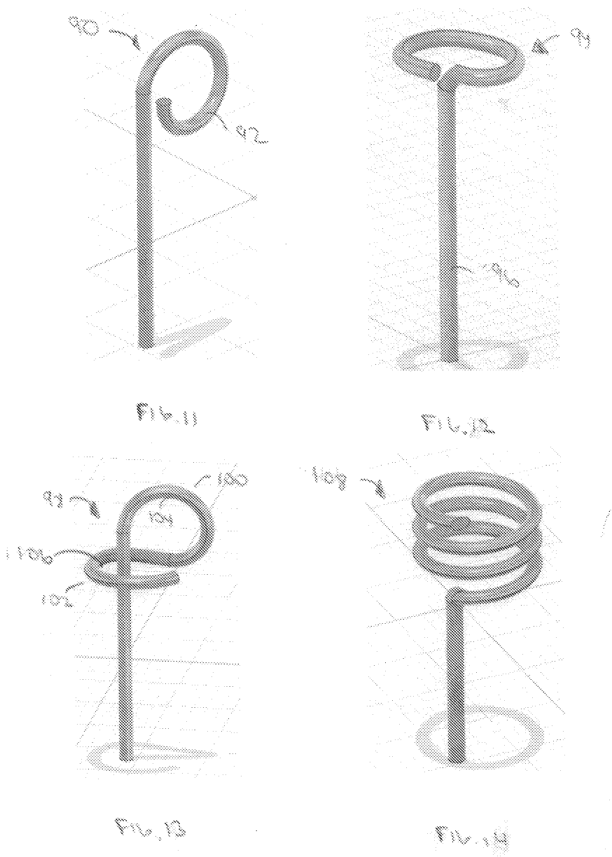

With reference to FIGS. 11-14, although the above embodiments disclose a catheter where the distal actuating portion has an inner curvature, the distal actuating portion can have other configurations. FIG. 11 illustrates distal actuating portion 90 with inner curvature 92 (consistent with the embodiments disclosed above). FIG. 12 illustrates distal actuating portion 94 that extends in a plane substantially perpendicular to the plane of the catheter shaft 96. FIG. 13 illustrates distal actuating portion 98 that includes at least two sections 100 and 102 with inner curvatures 104 and 106, respectively that extend in different planes. The distal actuating portion can include additional sections. FIG. 14 illustrates distal actuating portion 108 that has a spiral configuration. Such embodiments are only exemplary and the distal actuating portion of the catheter shaft can have other configurations.

In certain aspects, the catheter shaft can be fabricated or incorporate different materials such as, for example, a shape memory polymer (e.g. nitinol), nylon, latex or silicone. Further, the catheter can be impregnated with an antibacterial material such as copper or comprise a microscopic material texture rather than a substantially smooth texture to reduce catheter associated urinary tract infections. The catheter tip can comprise a Coude-tip for increased safety during insertion where the Coude curvature is oriented to facilitate catheter curling. In certain aspects, the balloon(s) are coated with silicone or similar material to mitigate or prevent balloon rupture.

Although catheters have been described herein with respect to urinary catheters, the catheters can also be used with respect to gastrostomy tubes, percutaneous drains, endovascular devices, endotracheal tubes, or other indwelling tubes or drains.

### EXAMPLE

A traction test, designed to measure the force needed to forcibly remove an inflated catheter, was performed. In order to mimic forced removal from the urethra, a plastic funnel was anchored to a ring stand. The funnel opening was lubricated, the catheter was fed through the lubricated opening, and subsequently inflated. A force meter was hooked onto the bottom of the catheter, and a force reading was recorded upon successful passage of the inflated catheter through the funnel opening. This test was performed on both a Foley catheter (16 French, 10 cc balloon, latex) and a catheter prototype as described herein with one drainage eyelet located at the tip of the catheter and four balloon segments. It should be noted that there was minimal radial expansion of the balloons due to limitations in prototyping capabilities but such minimal radial expansion did not affect the overall safety of the catheter prototype compared to the Foley catheter. 3 trials were conducted with the catheter prototype, and measures of <5 N, 5.76 N, and 6.1 N were recorded. With the Foley catheter, a force of >30 N was measured. This force reading is relatively consistent with those gathered through a study conducted at UCSF by Wu et al (Wu A K, Blaschko S D, Garcia M, McAninch J W, Aaronson D S. “Safer urethral catheters: how study of catheter balloon pressure and force can guide design;” BJU Int. 2012 April; 109 (7): 1110-4. Epub 2011 Aug. 22) measuring traction force required to dislodge Foley catheters in a similar plastic funnel model and cadavers (selected data included for comparison with Table I below).

TABLE I
Extraction Forces Measured by Wu et al.

| | | | Mean force |
|---|---|---|---|
| Foley catheter | | Balloon filling | required for |
| specifications | Model | volume, mL | dislodging, N |
| 16 French latex | Plastic Funnel | 10 | 41.37 |
| catheter with | Model | | |
| 5 mL balloon | | | |
| 16 French silicone | Plastic Funnel | 10 | 33.36 |
| catheter with | Model | | |
| 5 mL balloon | | | |
| 16 French latex | Cadaver | 10 | 45.82 |
| catheter with | | | |
| 5 mL balloon | | | |

The second test performed was a deployment force test, designed to measure the force needed to straighten the tip of the catheter prototype as described above. To test this, the catheter prototype was placed against a solid block of wood held in place. Following inflation of the catheter, the force required to straighten the tip of the catheter against the wooden block was measured. 3 trials were conducted, and a measure of <5 N was recorded for each.

Further traction testing was subsequently performed in ex vivo porcine bladders. A normal latex 16 Fr, 10 cc Foley catheter was inserted through an ex vivo porcine urethra into the bladder. The bladder was fixed in place and the force required to remove the catheter with an inflated balloon was measured, at approximately 27 N (the discrepancy noted compared to the aforementioned published studies can likely be explained by differences in tissue strength between adult humans and slaughter pigs, as well as the increased resistance to force provided by surrounding pelvic floor musculature and soft tissue that is present in cadavers but not in ex vivo bladders). Furthermore, once the balloon was forcefully moved distal to the prostatic urethra, the membranous urethra was grossly disrupted, as shown in the image below:

Gross membranous/pendulous urethral disruption with forced traditional catheter extraction

Additional testing using the same Foley catheter through just the prostatic urethra in another porcine ex-vivo model similarly showed a force requirement of ˜27 N for removal and caused an approximately 2 French dilation of the urethra in the process. When the same testing was performed with the catheter prototype, the amount of force required to remove the catheter prototype was between 10-11 N in two ex-vivo porcine bladders with no visible gross trauma or dilation occurring. The Table II below summarizes results from the ex vivo porcine bladder testing.

TABLE II
Extraction Force Measurements In Ex-Vivo Porcine Bladders

| | | Force | |
|---|---|---|---|
| | | required for | |
| Catheter | | dislodging, | |
| specifications | Model | N | Result |
| 16 French latex | Ex-vivo bladder | 27.4 | Gross urethral |
| standard Foley | and urethra | | disruption |
| catheter with | | | |
| 10 mL balloon | | | |
| 16 French latex | Ex-vivo | 27.9 | 2 French |
| standard Foley | prostatic urethra | | dilation |
| catheter with | | | of prostate |
| 10 mL balloon | | | |
| 20 French Atraumatic | Ex-vivo bladder | 10.4 | No visible |
| Catheter prototype | and urethra | | trauma |
| 20 French Atraumatic | Ex-vivo | 10.7 | No visible |
| Catheter prototype | prostatic urethra | | trauma |

These tests further demonstrate promise that the catheter prototype can be less injurious to patients.

Each of the disclosed aspects and embodiments of the present disclosure may be considered individually or in combination with other aspects, embodiments, and variations of the disclosure. Further, while certain features of embodiments and aspects of the present disclosure may be shown in only certain figures or otherwise described in the certain parts of the disclosure, such features can be incorporated into other embodiments and aspects shown in other figures or other parts of the disclosure. Along the same lines, certain features of embodiments and aspects of the present disclosure that are shown in certain figures or otherwise described in certain parts of the disclosure can be optional or deleted from such embodiments and aspects. Additionally, when describing a range, all points within that range are included in this disclosure. Further, unless otherwise specified, none of the steps of the methods of the present disclosure are confined to any particular order of performance. Furthermore, all references cited herein are incorporated by reference in their entirety.

What is claimed is:

1. A catheter having a non-actuated state and an actuated state and comprising:
a catheter shaft having central longitudinal axis extending therethrough, a distal actuating portion, a proximal portion, opposing first and second outer walls, and an inner wall between the opposing first and second outer walls;
a drainage section comprising a drainage lumen disposed between the inner wall and the first outer wall;
a drainage eyelet disposed in the drainage section;
an inflation section comprising an inflation lumen disposed between the inner wall and the second outer wall; and
a balloon at the distal actuating portion of the inflation section in fluid communication with the inflation lumen, the balloon offset from the central longitudinal axis of the catheter shaft and having a length and a width, the length of the balloon in an actuated state being greater than the length of the balloon in a non-actuated state, the width of the balloon remaining substantially the same in an actuated state and a non-actuated state.

2. The catheter of claim 1, wherein the length and width of the distal actuating portion opposite of the balloon remains substantially the same in a non-actuated state and an actuated state.

3. The catheter of claim 1, wherein the balloon comprises a plurality of balloons serially disposed at the distal actuating portion of the catheter shaft offset from the central longitudinal axis.

4. The catheter of claim 1, wherein the balloon comprises a single balloon.

5. The catheter of claim 1, wherein the balloon is disposed on an exterior surface of the inflation section at the distal actuating portion.

6. The catheter of claim 1, wherein the balloon is disposed within the interior of the inflation section at the distal actuating portion.

7. The catheter of claim 1, wherein the drainage eyelet is located proximal to the distal actuating portion, proximal to the distal actuating portion, or within the distal actuating portion.

8. The catheter of claim 1, wherein the drainage eyelet comprises a plurality of drainage eyelets.

9. The catheter of claim 1, wherein the eyelet comprises a static hinge.

10. The catheter of claim 1, wherein the balloon comprises semi-segmental sections, each semi-segmental section separated by an adjacent semi-segmental section by a septum.

11. The catheter of claim 10, wherein the drainage eyelet comprises a plurality of drainage eyelets, each of the plurality of eyelets comprising a static hinge disposed adjacent to a respective one of the semi-segmental sections of the balloon.

12. The catheter of claim 1, wherein the inner wall or another wall adjacent to the balloon has a thickness sufficient to promote axial lengthening of the balloon while limiting or preventing radial expansion of the balloon.

13. The catheter of claim 1, further comprising a first semi-rigid stiffener disposed adjacent to or embedded within a side of the balloon closest to the drainage section at the distal actuating portion, the first semi-rigid stiffener configured to promote axial lengthening of the balloon while limiting or preventing radial expansion of the balloon and promotes patency of the drainage lumen.

14. The catheter of claim 13, further comprising, a second semi-rigid stiffener disposed adjacent to or embedded within a side closest to an exterior surface of the balloon, the second semi-rigid stiffener configured to promote axial lengthening of the balloon while limiting or preventing radial expansion of the balloon.

15. The catheter of claim 1, further comprising an elastic or semi-rigid sheath disposed about or embedded within the inflation section that at least includes the distal actuating portion, an elastic or semi-rigid sheath disposed about the catheter shaft that at least includes the distal actuating portion, or disposed about the balloon itself, the elastic or semi-rigid sheath configured to promote axial lengthening of the balloon while limiting or preventing radial expansion of the balloon.

16. The catheter of claim 1, further comprising a spring, coil, ring, or other semi-rigid or elastic stiffener wrapped around or embedded within the inflation section at the distal actuating portion or wrapped around the balloon, the spring, coil, ring or other semi-rigid or elastic stiffener configured to promote axial lengthening of the balloon while limiting or preventing radial expansion of the balloon.

17. The catheter of claim 1, wherein the distal actuating portion comprises an inner curvature and an opposing outer curvature in an actuated state of the catheter.

18. The catheter of claim 1, wherein the distal actuating portion extends in a plane substantially perpendicular to a longitudinal axis of the catheter shaft.

19. The catheter of claim 1, wherein the distal actuating portion includes at least a first section having a first inner curvature, and a second section having a second inner curvature, the first inner curvature and the second inner curvature extending in different planes.

20. The catheter of claim 1, wherein the distal actuating portion has a spiral configuration.
